(19) United States
(12) Patent Application Publication
(10) Pub. No.: US 2023/0320659 A1
(43) Pub. Date: Oct. 12, 2023

(54) TECHNIQUES FOR NON-INVASIVELY MONITORING DEHYDRATION

(71) Applicant: THE REGENTS OF THE UNIVERSITY OF MICHIGAN, Ann Arbor, MI (US)

(72) Inventors: Jenna WIENS, Ann Arbor, MI (US); Kathleen SIENKO, Ann Arbor, MI (US)

(73) Assignee: THE REGENTS OF THE UNIVERSITY OF MICHIGAN, Ann Arbor, MI (US)

(21) Appl. No.: 18/028,106

(22) PCT Filed: Oct. 22, 2021
(86) PCT No.: PCT/US2021/056142

§ 371 Date: Mar. 23, 2023

Related U.S. Application Data
(60) Provisional application No. 63/104,572, filed on Oct. 23, 2020.

(51) Int. Cl. A61B 5/00; A61B 5/024; A61B 5/11

(52) U.S. Cl. CPC A61B 5/4875; A61B 5/7267; A61B 5/7257; A61B 5/024; A61B 5/6801; A61B 5/0002; A61B 5/1116

(57) ABSTRACT

A computer-implemented method is presented for quantifying hydration in a subject. The method includes: receiving a heart rate signal indicative of heart rate of the subject; extracting features from the heart rate signal, where one or more of the extracted features include a frequency domain representation of the heart rate signal; constructing a feature vector from the extracted features; and quantifying hydration status of the subject as a percent of body weight of the subject by classifying the feature vector using machine learning, where percentages of body weight are expressed in increments on the order of one percent or less.

### CROSS-REFERENCE TO RELATED APPLICATIONS

This application is a PCT International Application and claims the benefit of U.S. Provisional Application No. 63/104,572, filed on Oct. 23, 2020. The entire disclosure of the above application is incorporated herein by reference.

### FIELD

The present disclosure relates to techniques for non-invasively monitoring hydration status in a subject.

### BACKGROUND

Dehydration in athletes and active individuals is associated with heat-related injuries ranging from fatigue and cramps to heat stroke and death. Fluid losses as small as 2% of body weight can negatively affect physical performance. Less effective thermoregulatory responses also put children and the elderly at high risk of developing heat illnesses associated with dehydration.

Typical measures of hydration status, such as body weight, urine specific gravity, blood plasma levels, and bioelectrical impedance may be inconvenient (e.g., equipment access), invasive (e.g., drawing blood), costly (e.g., bioelectrical impedance systems and/or trained personnel) or location impeded. An alternative strategy uses steady-state, orthostatic measurements to detect fluid loss. For example, heart rate is measured by palpation after the subject lies supine for two minutes and then again after the subject stands for one minute. With normal fluid levels, the steady-state, standing heart rate is approximately 11 beats per minute (bpm) greater than the steady-state supine heart rate. With low fluid levels, the difference is 30 bpm or more. As prescribed, this method does not allow unobtrusive monitoring within or outside of a clinical setting, and it fails to capture the heart rate transients during posture changes. Although it can detect moderate to large fluid losses, it is unreliable for detecting low fluid losses. Therefore, it is desirable to develop improved and non-invasive techniques for monitoring and measuring hydration status in a subject.

This section provides background information related to the present disclosure which is not necessarily prior art.

### SUMMARY

This section provides a general summary of the disclosure, and is not a comprehensive disclosure of its full scope or all of its features.

In one aspect, a computer-implemented method is presented for quantifying hydration in a subject. The method includes: receiving a heart rate signal indicative of heart rate of the subject; extracting features from the heart rate signal, where one or more of the extracted features include a frequency domain representation of the heart rate signal; constructing a feature vector from the extracted features; and quantifying hydration status of the subject as a percent of body weight of the subject by classifying the feature vector using machine learning, where percentages of body weight are expressed in increments on the order of one percent or less.

In one example, the feature vector includes mean of the heart rate signal, median of the heart rate signal, a maximum of the heart rate signal, a minimum of the heart rate signal, standard deviation of the heart rate signal and a series of Fourier coefficients representing the heart rate signal.

The change in hydration of the subject may be quantified by classifying the feature vector into a class selected from a group consisting of hydrated, dehydrated by one percent of body weight and dehydrated by two percent of body weight.

In another aspect, the computer-implemented method for quantifying hydration in a subject include: measuring heart rate of the subject using a heart rate sensor; receiving a signal indicative of heart rate of the subject from the heart rate sensor; extracting features from the signal; constructing a feature vector from the extracted features, where one or more of the extracted features include a frequency domain representation of the heart rate signal; detecting a change in posture of the subject; and quantifying a change in hydration of the subject in response to detecting a change in posture of the subject, where the change in hydration is quantified by classifying the feature vector using machine learning.

In one embodiment, the change in posture of the subject is detected by measuring inclination of torso of the subject in relation to an upright position.

In yet another aspect, the computer-implemented method for quantifying hydration in a subject includes: measuring heart rate of the subject using a heart rate sensor; detecting a change in posture of the subject; receiving a signal indicative of heart rate of the subject before and after the change in posture of the subject; extracting features from the signal; constructing a feature vector from the extracted features, where the change in posture is an element of the feature vector; and quantifying a change in hydration of the subject in response to detecting a change in posture of the subject, where the change in hydration is quantified by classifying the feature vector using machine learning.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram depicting a subject equipped with a system for monitoring hydration.

FIG. 2 is a block diagram of components comprising the system for monitoring hydration.

FIG. 3 is a flowchart depicting an example embodiment for quantifying hydration of a subject.

FIGS. 4A-4K are graphs illustrating experimental results for different individuals in a hydrated pre-exercise state, a hydrated post-exercise state, and a dehydrated post-exercise state.

FIG. 5 is a flowchart depicting another example embodiment for quantifying hydration of a subject.

Corresponding reference numerals indicate corresponding parts throughout the several views of the drawings.

### DETAILED DESCRIPTION

Example embodiments will now be described more fully with reference to the accompanying drawings.

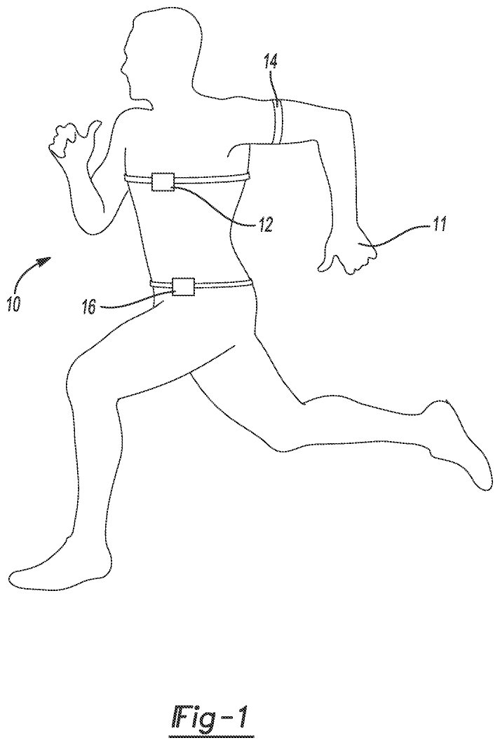

FIG. 1 illustrates a system for monitoring hydration 10 in a subject 11 according to this disclosure. The system 10 is comprised generally of a heart rate sensor 12, an orientation sensor 14 and a data management device 16. In the example embodiment, the heart rate monitor 12, the orientation sensor 14 and the data management device are three separate devices. Each of the three devices 12, 14, 16 are configured to be worn or otherwise attached to the subject 11. In other embodiments, the heart rate monitor 12 and the orientation sensor 14 are worn by the subject while the data management device 16 (e.g., a mobile phone) is carried by the subject. Alternatively, the data management device 16 may be positioned proximate to the subject, for example on exercise equipment being used by the subject. In yet other embodiments, one or more of the devices may be integrated into a common housing. For example, the heart rate monitor and the orientation sensor may be integrated into one common housing while the data management device 16 remains a separate device. In another example, all three devices are integrated into one common housing. Other configurations for these components are contemplated by this disclosure.

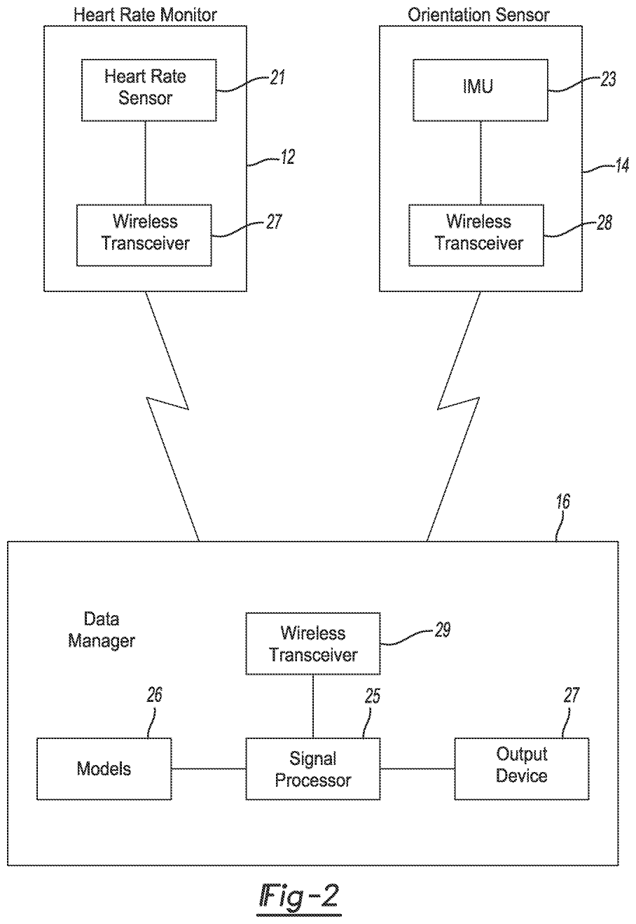

FIG. 2 further illustrates each of the components of the system 10. In this example embodiment, the system components include a heart rate sensor 12, an orientation sensor 14, and a data management device 16. Each component 12, 14, 16 is configured to wirelessly communicate with each other in accordance with a wireless communication protocol, such as Bluetooth BLE. In this regard, each component 12, 14, 16 is equipped with a wireless transceiver 29. Other types of wireless communication are contemplated by this disclosure, including infrared communication.

The heart rate monitor 12 is comprised of a heart rate sensor 21 and a wireless transceiver 29. The heart rate sensor 21 is designed to capture a heart rate signal indicative of the heart rate of the subject. The heart rate sensor 21 is interfaced with the wireless transceiver 29. The wireless transceiver in turn communicates the heart rate signal to the data management device 16. In the example embodiment, the heart rate monitor is a H10 heart rate monitor commercially available from Polar Electro. Similar types of heart rate monitors are envisioned by this disclosure.

In one example, the orientation sensor 14 includes an inertial measurement unit (IMU) 23 interfaced with a wireless transceiver 29. The IMU 23 is designed to report the orientation of the subject, for example relative to an upright position as defined by a gravity vector. More specifically, the orientation sensor 14 reports the inclination of the torso of the subject in relation to an upright position. In this way, the system can detect a change in the posture of the subject, for example from standing upright to a prone position. In other examples, the orientation sensor 14 may use one or more accelerometers, gyroscopes or other types of motion sensors. Likewise, the wireless transceiver 29 communicates the orientation of the subject to data management device 16. In the example embodiment, the orientation sensor is Trigno EKG biofeedback sensor commercially available from Delsys Inc. Similar types of orientation sensors are envisioned by this disclosure.

The data management device 16 is comprised of a signal processor 25, a set of models residing in a non-transitory data store 26, an output device 27 (e.g., a speaker and/or display) and a wireless transceiver 29. The data management device 16 is configured to receive the heart rate signal from the heart rate monitor 12. The data management device 16 is also configured to receive data indicating the orientation of the subject from the orientation sensor 14. In the example embodiment, the data management device is OptimEye S5 data manager commercially available from Catapult Inc. Similar types of data managers are envisioned by this disclosure.

Based on this data, the signal processor 25 is designed to monitor and quantify hydration of the subject using machine learning as will be further described below. In one embodiment, the change in hydration status is quantified in increments on the order of one percent or less of the body weight of the subject. For example, the hydration status may be classified as hydrated, dehydrated by one percent, dehydrated by two percent or dehydrated by more than two percent. These classification are merely illustrative as more or less classes are envisioned as well as more granular increments. The signal processor 25 may cooperate with the output device 27 to notify the subject or another person of the hydration status. In one example, hydration status is displayed on a display device. Audible and/or other visual notifications are also contemplated by this disclosure.

For each of these devices 12, 14, 16, it is to be understood that only the relevant components are discussed in relation to FIG. 2, but that other components within each device (e.g., power source) may be needed to control and manage the overall operation of the system.

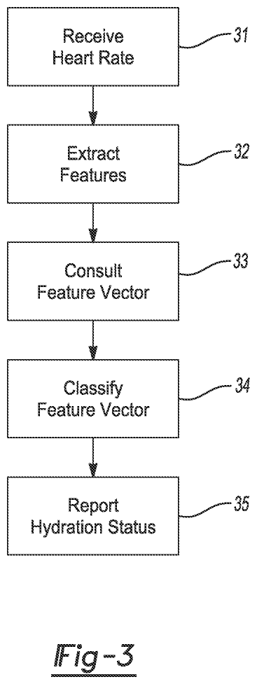

FIG. 3 depicts an example method for quantifying hydration of a subject using the system 10 described above. As a starting point, a heart rate signal indicative of heart rate of the subject is received at 31 by the signal processor. The heart rate signal is a time series captured over a period of time by a heat rate monitor affixed to the subject. In this example, hydration is quantified solely using a heart rate signal of the subject.

Features are extracted from the heart rate signal as indicated at 32. Features extracted from the heart rate signal may include typical statistical descriptors including but not limited to mean, median, quantile, and standard deviation. Extracted features may also include energy ratios, correlations (e.g., autocorrelation) and other statistics. Of note, one or more of the extracted features from the heart rate signal include a frequency domain representation of the heart rate signal or a portion thereof. The frequency domain representation of the heart rate signal can be obtained by applying a Fourier transform, a wavelet transform or other known transform which yield a frequency domain representation of the heart rate signal.

Next, a feature vector is constructed at 33 from the extracted features. In one example embodiment, the feature vector is comprised of the mean of the heart rate signal, the median of the heart rate signal, the maximum of the heart rate signal, the minimum of the heart rate signal, the standard deviation of the heart rate signal and a series of coefficients representing the Fourier transform of the heart rate signal. In another example embodiment, the feature vector is comprised of mean of the heart rate signal, the median of the heart rate signal, a quantile value of the heart rate signal, the standard deviation of the heart rate signal and a series of coefficients representing a continuous wavelet transform of the heart rate signal. It is readily understood from these examples that the feature vector can include different combinations of the features contemplated by this disclosure. It is also envisioned that the heart rate signal without any feature extraction could also serve as input to the classification process.

To quantify the hydration status of the subject, the feature vector is classified at 34 using machine learning. For example, the feature vector is classified into a class selected from a group consisting of hydrated, dehydrated by one percent of body weight, dehydrated by two percent of body weight or dehydrated by more than two percent of body weight. It is understood that the trained models may be particular to an individual or derived from a broader population. In the example embodiment, the feature vector may be classified using random decision forests. Other types of neural networks are contemplated by this disclosure including logistic regression and recurrent neural networks. Moreover, the broader aspects of this disclosure are not limited to neural networks but may extend to other types of machine learning.

In order to train the machine learning model, the necessary feature vector was calculated from a specified window of the heart rate signal. Each individual completes two exercise sessions—one without fluids (i.e., dehydrated), one with replenishing fluids (i.e., hydrated). Multiple heart rate signals are extracted for each individual from both sessions following exercise. The model then outputs a probability estimate of the individual being dehydrated given this feature vector. In order to test the generalizability of the models across individuals, models are trained using examples from all individuals except one, and then the performance of the model is examined on examples from the held-out individual. This procedure is repeated multiple times until every individual has been held out of the test set exactly once. As proof of concept, the performance of two types of model and input pairs were examined: 1) non-deep models, such as logistic regression and random forests (ensembles of decision trees) which take in as input the feature vector described above, and 2) recurrent neural network based methods which take in the unprocessed heart rate signal. In order to evaluate the models, the area under the receiver operating characteristics (AUROC) is calculated, which measures the discriminative ability of a binary classifier. An AUROC of 0.5 represents a random classifier, while a perfect classifier achieves an AUROC of 1.0.

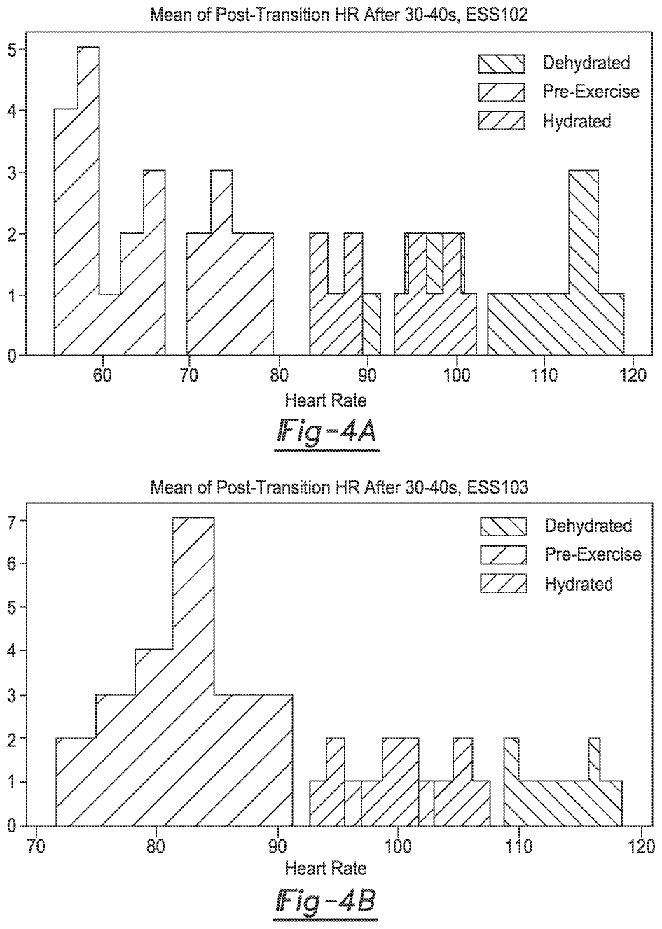

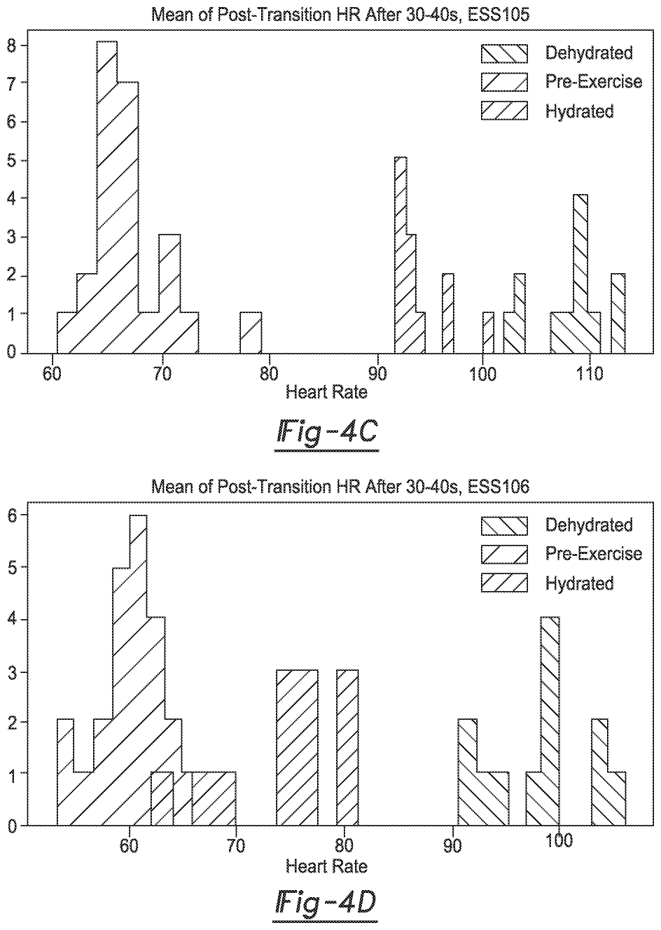

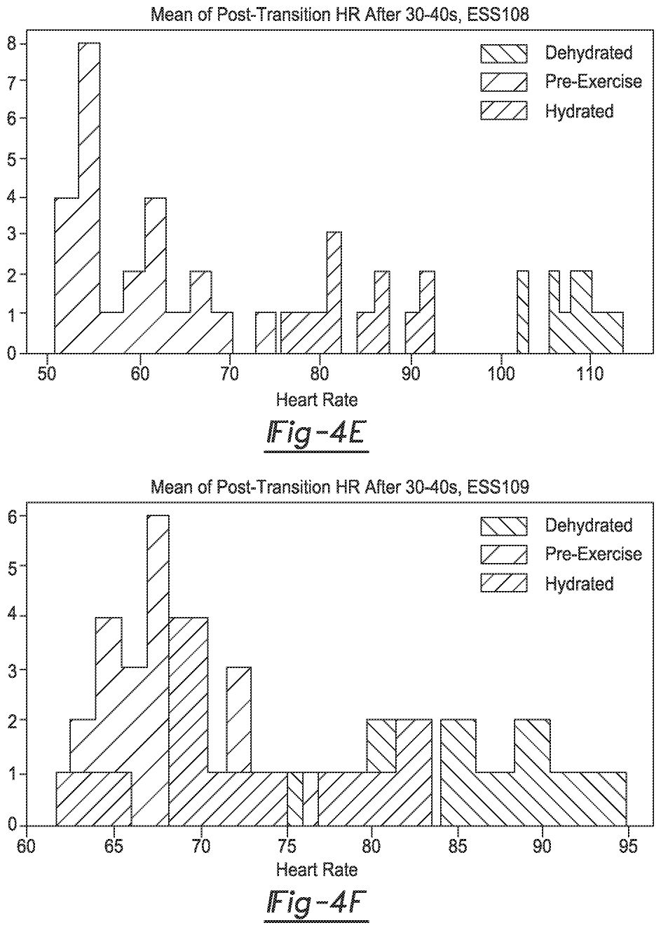

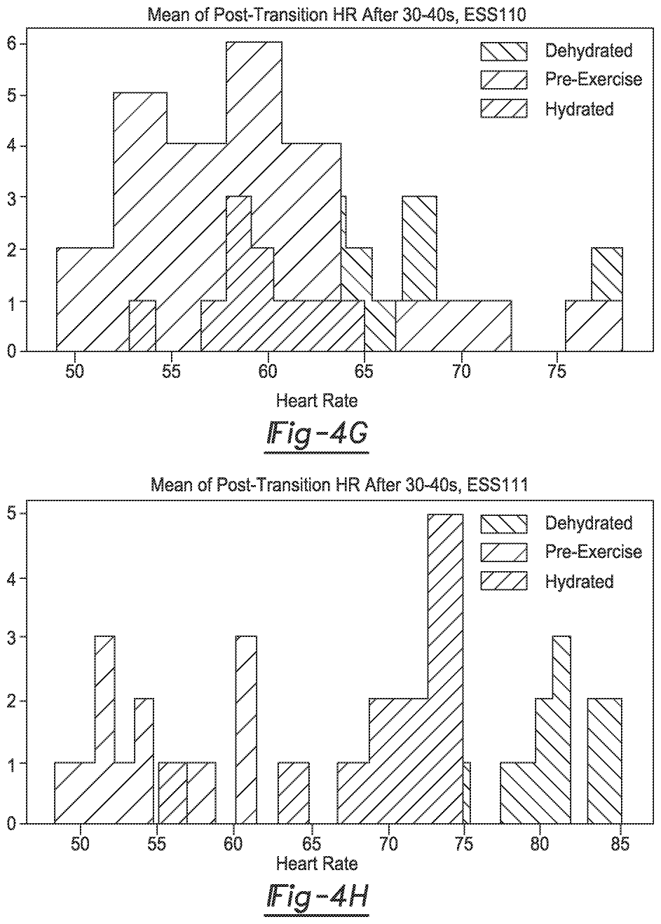

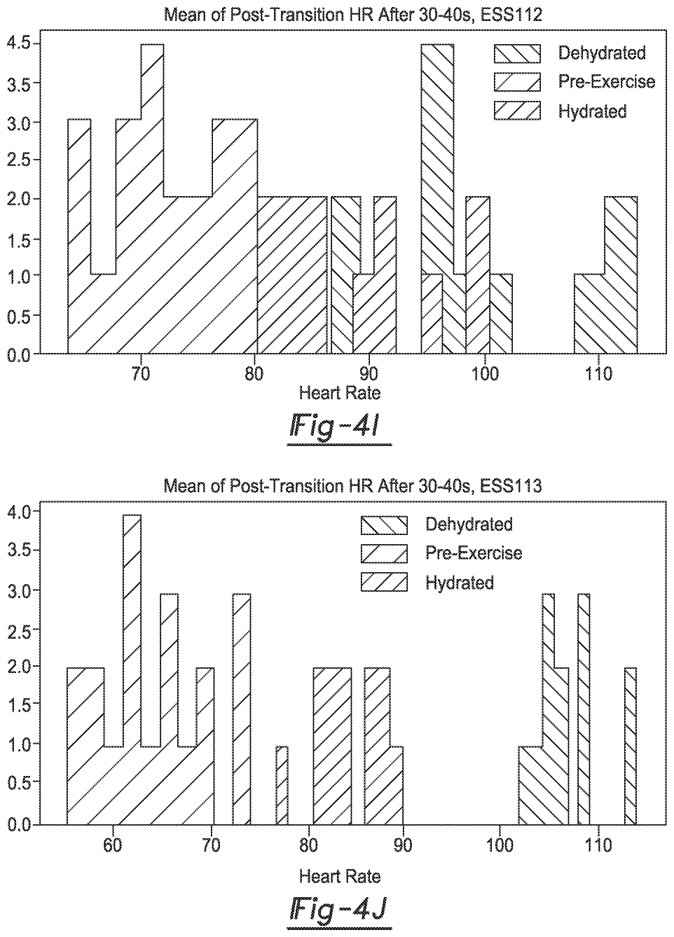

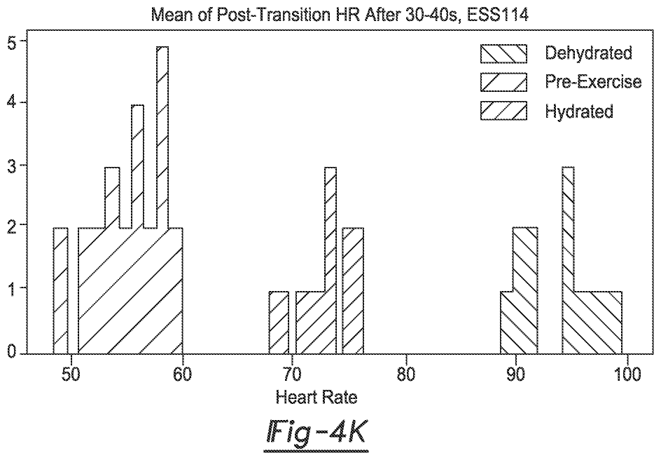

Iterative testing was performed on 12 separate individuals who have undergone the protocol using the experimental procedure described above. The full feature vector is calculated at a time of standing (after performing a postural change), and used as input to the model. Over the testing of different individuals, the average AUROC is 0.83. A simpler feature vector having the mean value of a 10 second heart rate signal 30-40 seconds after some postural change has occurred is also considered. Using this minimal feature as input results in an average AUROC of 0.950. FIGS. 4A-4K shows how the mean heart rate changes for different individuals changes as they are: 1) hydrated pre-exercise, 2) hydrated post-exercise, and 3) dehydrated post-exercise for multiple different windows of heart rate. Using this feature results in a good separation for a majority of individuals and reveals a noticeable pattern: exercise increases heart rate, but dehydration in combination with exercise results in an even larger increase.

With continued reference to FIG. 3, the quantified hydration status of the subject is reported as indicated at 35. In one example, the subject is classified as being hydrated, dehydrate by one percent of their body weight or dehydrated by two percent of their body weight, and the quantified status is displayed on a device associated with the subject, such as a phone, watch or another mobile computing device. Additionally or alternatively, an alert may be triggered when the subject's status is indicated as being dehydrated. The alert may take may different forms including flashing light, audible sounds or tactile feedback. It is also envisioned that the alert may be transmitted by the system 10 to a third party device located remotely from the subject.

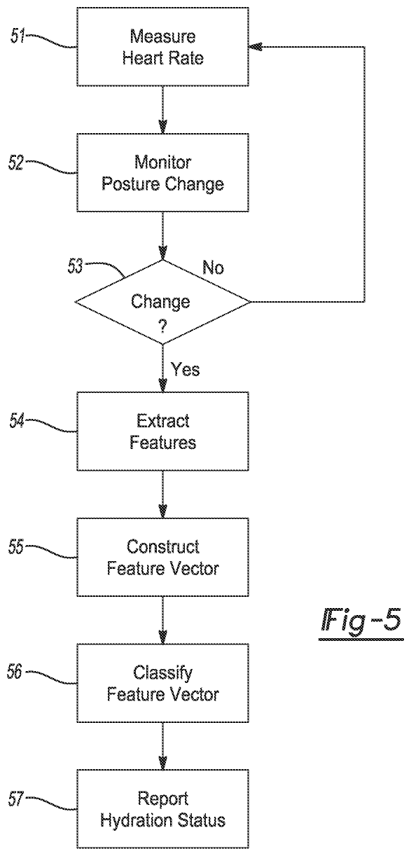

FIG. 5 depicts another example method for quantifying hydration of a subject using the system 10 described above. Again, a heart rate signal for the subject is measured as indicated at 51, where the heart rate signal is a time series captured over a period of time by a heat rate monitor affixed to the subject.

In this embodiment, quantifying hydration is triggered by a change in the posture of the subject. The posture of the subject is therefore monitored at 52, for example using an orientation sensor affixed to the subject. In one example, the inclination of torso of the subject is monitored in relation to an upright position. In another example, the position of the subjects head is monitored in relation to the subject's heart. These changes is posture may occur, for example when the subject is touching their toes or otherwise bent over during exercise. In yet another example, posture of the subject may change when a subject in a bed (e.g., at a hospital) sits up or lays down. These examples are merely illustrative of the different activities which may cause a change in the subject's posture. In the absence of a change in posture, the heart rate of the subject continues to be monitored as indicated at 53.

When the change in posture exceeds a threshold, hydration status can be quantified. In the example embodiment, hydration status is quantified when the inclination of torso is greater than 30 degrees offset from an upright position. To quantify hydration status, features are extracted from the heart rate signal at 54 and a feature vector is constructed from the extracted features at 55. In this embodiment, extracted features may account for postural change, such as a feature defined as the mean heart rate in intervals after the postural change subtracted by the baseline heart rate right before the postural change occurs. For example, one element in the feature vector could be the average heart rate 30-40 seconds after the postural change subtracted by the average heart rate 15-5 seconds before the postural change occurs. Additionally, the feature vector may include features that describe the postural change. Such features may include but are not limited the total time before the postural change and/or the change in pitch that occurs with the postural change. Another feature could be a multivariate time-series representation of the data which includes the heart rate signal and the pitch signal during a postural change could be used as input to a neural network model in order to learn how the combination of heart rate and posture can detect dehydration.

In one embodiment, the feature vector is comprised of a first difference between mean heart rate before postural transition (e.g., 5-15 seconds before postural change) and mean heart rate after the postural transition during a first successive time period (e.g., 10-20 seconds after postural change), a second difference between mean heart rate before postural transition and mean heart rate after the postural transition during a second successive time period (e.g., 20-30 seconds after postural change), a third difference between mean heart rate before postural transition and mean heart rate after the postural transition during a third successive time period (e.g., 30-40 seconds after postural change), a fourth difference between mean heart rate before postural transition and mean heart rate after the postural transition during a fourth successive time period (e.g., 40-50 seconds after postural change), a fifth difference between mean heart rate before postural transition and mean heart rate after the postural transition during a fifth successive time period (e.g., 50-60 seconds after postural change), a sixth difference between mean heart rate before postural transition and mean heart rate after the postural transition during a sixth successive time period (e.g., 60-70 seconds after postural change). The example feature vector considers splitting the post-transition heart rate signal into six equally sized intervals to take the average over. More or less post transition time periods can be used to construct the feature vector. Note that the duration of the time period and the specific number of segments to take the average heart rate over post-transition can be varied. Other variants for the feature vector are envisioned by this disclosure.

To quantify the hydration status of the subject, the feature vector is classified at 56 using machine learning in the manner described above. In the example embodiment, the feature vector is classified into a class selected from a group consisting of hydrated, dehydrated by one percent of body weight, dehydrated by two percent of body weight or dehydrated by more than two percent of body weight. The class having the highest likelihood is then report at 57 as the hydration status for the subject.

This approach was evaluated on different sets of postural changes. In order to identify whether one can identify dehydration using shorter postural changes, such as 30-second toe-touches or 30-second “tired runner” poses, this disclosure considered training using all postural movements, but evaluating on: 1) only supine to standing movements, 2) only toe-touches, and 3) all 30-second postural movements. Using the iterative testing procedure described above and using a logistic regression model, this method was able to achieve an average AUROC of: 0.865 for evaluating on toe-touches, 0.809 for evaluating on 30-second postural movements, and 0.844 for supine to standing postural movements.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for quantifying hydration in a subject, comprising:
receiving, by a signal processor, a heart rate signal indicative of heart rate of the subject;
extracting, by the signal processor, features from the heart rate signal, where one or more of the extracted features include a frequency domain representation of the heart rate signal;
constructing, by the signal processor, a feature vector from the extracted features; and
quantifying hydration status of the subject as a percent of body weight of the subject by classifying the feature vector using machine learning, where percentages of body weight are expressed in increments on the order of one percent or less.

2. The method of claim 1 further comprises capturing the heart rate signal using a sensor affixed to the subject.

3. The method of claim 1 wherein extracting features from the heart rate signal further comprises applying one of a Fourier transform or a wavelet transform to the heart rate signal.

4. The method of claim 1 wherein the feature vector includes mean of the heart rate signal, median of the heart rate signal, a maximum of the heart rate signal, a minimum of the heart rate signal, standard deviation of the heart rate signal and a series of Fourier coefficients representing the heart rate signal.

5. The method of claim 1 wherein quantifying a change in hydration of the subject further comprises classifying the feature vector into a class selected from a group consisting of hydrated, dehydrated by one percent of body weight and dehydrated by two percent of body weight.

6. The method of claim 1 further comprises classifying the feature vector using random decision forests.

7. The method of claim 1 further comprises classifying the feature vector using a recurrent neural network.

8. The method of claim 1 further comprises displaying the hydration status of a display device.

9. The method of claim 1 further comprises transmitting an alert to another device located remotely from the signal processor.

10. A computer-implemented method for quantifying hydration in a subject, comprising:
measuring heart rate of the subject using a heart rate sensor;
receiving, by a signal processor, a signal indicative of heart rate of the subject from the heart rate sensor;
extracting, by the signal processor, features from the signal;
constructing, by the signal processor, a feature vector from the extracted features, where one or more of the extracted features include a frequency domain representation of the heart rate signal;
detecting a change in posture of the subject; and
quantifying a change in hydration of the subject in response to detecting a change in posture of the subject, where the change in hydration is quantified by classifying the feature vector using machine learning.

11. The method of claim 10 further comprises quantifying the change in posture, wherein the change in posture is an element of the feature vector.

12. The method of claim 10 wherein detecting a change in posture of the subject comprises measuring inclination of torso of the subject in relation to an upright position.

13. The method of claim 10 wherein extracting features from the heart rate signal further comprises applying one of a Fourier transform or a wavelet transform to the heart rate signal.

14. The method of claim 10 wherein quantifying a change in hydration of the subject further comprises classifying the feature vector into a class selected from a group consisting of hydrated, dehydrated by one percent and dehydrated by two percent.

15. The method of claim 10 further comprises classifying the feature vector using random decision forests.

16. A computer-implemented method for quantifying hydration in a subject, comprising:
measuring heart rate of the subject using a heart rate sensor;
detecting a change in posture of the subject;
receiving, by a signal processor, a signal indicative of heart rate of the subject before and after the change in posture of the subject;
extracting, by the signal processor, features from the signal;
constructing, by the signal processor, a feature vector from the extracted features, wherein the change in posture is an element of the feature vector; and
quantifying a change in hydration of the subject in response to detecting a change in posture of the subject, where the change in hydration is quantified by classifying the feature vector using machine learning.

17. The method of claim 16 wherein detecting a change in posture of the subject comprises measuring inclination of torso of the subject in relation to an upright position.

18. The method of claim 16 wherein quantifying a change in hydration of the subject further comprises classifying the feature vector into a class selected from a group consisting of hydrated, dehydrated by one percent and dehydrated by two percent.

19. The method of claim 16 further comprises classifying the feature vector using random decision forests.
